SEQUENCE LISTING (2 sequences)

SEQ ID NO: 1 - DNA, 21 bp, Artificial sequence
  Synthetic oligonucleotide
gtgctggacaaatggttgctg

SEQ ID NO: 2 - DNA, 22 bp, Artificial sequence
  Synthetic oligonucleotide
aggaggatgcttctgagccttc

(19) United States
(12) Patent Application Publication
(10) Pub. No.: US 2005/0267027 A1
(43) Pub. Date: Dec. 1, 2005

(54) Use of erythropoietin for treatment of cancer

(76) Inventors: Karen M. Lounsbury, Essex Junction, VT (US); Cheung Wong, South Burlington, VT (US); Sarah A. Hale, Essex Junction, VT (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 11/093,177

(22) Filed: Mar. 28, 2005

Related U.S. Application Data
(60) Provisional application No. 60/559,479, filed on Apr. 5, 2004.

(51) Int. Cl. A61K 38/18

(52) U.S. Cl. 514/12

(57) ABSTRACT

A method for treating cancer is provided. The method involves administering to subjects in need of such treatment an effective amount of erythropoietin of an analog thereof effective to inhibit angiogenesis in a tumor. Also provided are methods to reduce HIF-1α protein levels and/or VEGF expression, particularly in tumors.

### RELATED APPLICATIONS

This application claims the benefit under 35 U.S.C. § 119(e) of U.S. provisional patent Application Ser. No. 60/559,479, filed on Apr. 5, 2004, the entire contents of which are hereby expressly incorporated by reference.

### FIELD OF THE INVENTION

Methods for treating cancer are provided. The methods involve administering to subjects in need of such treatment an effective amount of erythropoietin of an analog thereof effective to inhibit angiogenesis in a tumor. Also provided are methods to reduce HIF-1α protein levels and/or VEGF expression, particularly in tumors.

### BACKGROUND OF THE INVENTION

Angiogenesis is a key factor in the growth and metastasis of ovarian tumors (Folkman J., J. Natl. Cancer Institute, 2000, 92: 94-5; Semenza G L., Cancer and Metastasis Reviews, 2000, 19:59-65; Rofstad E K., Int J Radiat Biol, 2000, 76:589-605). The hypoxic core of the tumor initiates a signaling cascade that leads to expression of pro-angiogenic factors. Vascular endothelial growth factor (VEGF) is a potent angiogenic growth factor whose expression is regulated by hypoxia through the transcription factor hypoxia inducible factor-1 (HIF-1) (Semenza G L., J Appl Physiol, 2000, 88:1474-80). HIF-1 is comprised of a constitutively expressed β subunit and an α subunit that is sensitive to cellular oxygen tension (Lee J W, et al., Exp Mol Med, 2004, 36:1-12).

In normoxia, HIF-1α is targeted for ubiquitin-directed proteosomal degradation, through its interaction with the von Hippel Lindau tumor suppressor (VHL) (Lee J W, et al., Exp Mol Med, 2004, 36:1-12). Because oxygen-dependent prolyl hydroxylation is required for VHL binding to HIF-1α, hypoxia leads to HIF-1α stabilization, thus promoting transcription of target genes including VEGF and erythropoietin, as well as genes involved in glycolysis, adaptation to pH and apoptosis (Lee J W, et al., Exp Mol Med, 2004, 36:1-12).

High levels of VEGF and HIF-1α have been correlated with aggressive tumor types, in particular, gynecologic malignancies (Semenza G L., Nature Reviews Cancer, 2003, 3:721-732; Wong C, et al., Gynecol. Oncol., 2003, 91:513-517). In addition, our laboratory has reported a strong correlation between high expression of VEGF and HIF-1α in advanced stages of ovarian cancer (Wong C, et al., Gynecol. Oncol., 2003, 91:513-517). As such, HIF-1α is an attractive target for the development of therapies to combat VEGF-mediated angiogenesis in ovarian cancer particularly and other cancers more generally.

### SUMMARY OF THE INVENTION

It has now been discovered that erythropoietin reduces hypoxia-induced HIF-1α protein levels in tumor cells, probably by inhibiting hypoxia-induced stabilization of the HIF-1α protein or by decreased translation of HIF-1α. In addition, it has been discovered that erythropoietin inhibits vascular endothelial growth factor (VEGF) expression in tumor cells during hypoxia. Therefore, erythropoietin has anti-angiogenic activity, and is useful in the treatment of tumors. Given the prior understanding of erythropoietin activity, it was entirely unexpected that erythropoietin would have the activities described herein.

According to one aspect of the invention, methods of treating cancer in a subject in need of such treatment is provided. The methods include administering to the subject an amount of erythropoietin (EPO) or EPO analog effective to inhibit angiogenesis in a tumor in the subject. In some embodiments, the tumor is a breast cancer, an ovarian cancer, a colon cancer, a brain cancer, a cervical cancer, a lung cancer, a hematological neoplasm, a liver cancer, a lymphoma, a pancreatic cancer, a prostate cancer, a sarcoma, or a skin cancer. In other embodiments, the tumor is a solid tumor growth, a tumor metastasis, or a precancerous lesion.

In further embodiments, the EPO or EPO analog administered locally or is administered systemically. In preferred embodiments, the subject is otherwise free of symptoms calling for treatment with EPO or an EPO analog.

In some embodiments, the EPO or EPO analog is administered in combination with one or more anti-cancer compounds. In other embodiments the EPO or EPO analog is administered in combination with surgery to remove the tumor, or is administered to a patient who has had surgery to remove the tumor. In still other embodiments, the EPO or EPO analog is administered in combination with one or more anti-angiogenic compounds.

According to another aspect of the invention, methods for reducing hypoxia-induced HIF-1α protein levels are provided. The methods include contacting a cell with an amount of erythropoietin (EPO) or EPO analog effective to reduce hypoxia-induced HIF-1α protein levels. In certain embodiments, the EPO or EPO analog inhibits hypoxia-induced HIF-1α stabilization, increases HIF-1α destabilization, and/or decreases HIF-1α translation. In preferred embodiments, the HIF-1α protein levels are reduced at least about 50%.

According to yet another aspect of the invention, methods for reducing hypoxia-induced HIF-1α protein levels in a subject are provided. The methods include administering to the subject an amount of erythropoietin (EPO) or EPO analog effective to reduce hypoxia-induced HIF-1α protein levels. In some preferred embodiments, the subject is otherwise free of symptoms calling for treatment with EPO or an EPO analog.

In certain embodiments, the EPO or EPO analog is administered locally or is administered systemically. In other embodiments, the EPO or EPO analog inhibits hypoxia-induced HIF-1α stabilization, increases HIF-1α destabilization, and/or decreases HIF-1α translation. In preferred embodiments, the HIF-1α protein levels are reduced at least about 50%.

According to a further aspect of the invention, methods for inhibiting hypoxia-induced VEGF expression are provided. The methods include contacting a cell with an amount of erythropoietin (EPO) or EPO analog effective to inhibit the expression of VEGF. In certain embodiments, the EPO or EPO analog inhibits VEGF transcription or translation. In preferred embodiments, the VEGF expression levels are reduced at least about 50%.

According to still another aspect of the invention, methods for inhibiting hypoxia-induced VEGF expression in a subject are provided. The methods include administering to the subject an amount of erythropoietin (EPO) or EPO analog effective to inhibit the expression of VEGF. In some preferred embodiments, the subject is otherwise free of symptoms calling for treatment with EPO or an EPO analog.

In certain embodiments, the EPO or EPO analog is administered locally or is administered systemically. In other embodiments, the EPO or EPO analog inhibits VEGF transcription or translation. In preferred embodiments, the VEGF expression levels are reduced at least about 50%.

In another aspect of the invention, pharmaceutical preparations are provided. The pharmaceutical preparations include erythropoietin (EPO) and/or at least one EPO analog, at least one anti-cancer compound and/or at least one anti-angiogenesis compound, and a pharmaceutically acceptable carrier.

According to still another aspect of the invention, kits are provided that include a first container housing erythropoietin (EPO) and/or at least one EPO analog, and a second container housing at least one anti-cancer compound and/or at least one anti-angiogenesis compound. In some embodiments, the first container and/or second container further contain a pharmaceutically acceptable carrier. In other embodiments, the kits also include a third container containing a pharmaceutically acceptable carrier.

The use of the foregoing compositions in the preparation of medicament also is provided. In preferred embodiments, the medicament is useful in the treatment of cancer.

Each of the limitations of the invention can encompass various embodiments of the invention. It is, therefore, anticipated that each of the limitations of the invention involving any one element or combinations of elements can be included in each aspect of the invention.

These and other aspects of the invention will be described in greater detail below.

### BRIEF DESCRIPTION OF THE DRAWINGS

FIG. 1 shows that EPO inhibits HIF-1α protein levels induced by hypoxia. SK-OV-3 cells were treated for 16 h with the indicated doses of EPO under normoxia or hypoxic (0.1% or 2% O₂) conditions. FIG. 1A, Cell lysates (25 μg) were analyzed by immunoblot using an antibody recognizing HIF-1α. FIG. 1B, Summary data of the effects of EPO on HIF-1α detected by immunoblot and expressed as a percent of the hypoxic response; *, p<0.05, ** p<0.01, ***p<0.001, n=4. FIG. 1C, Cells were analyzed by immunofluorescence using an antibody recognizing HIF-1α (red). Nuclei were stained with YoYo-1 (green). FIG. 1D, RNA was extracted from SK-OV-3 cells and HIF-1α mRNA was amplified using RT-PCR. Shown is an agarose gel of amplified HIF-1α.

FIG. 2 shows that EPO decreases hypoxia-induced transcription of VEGF in SK-OV-3 cells. FIG. 2A, Cells were treated with EPO for 48 h under normoxic or hypoxic (0.1% or 2% O₂) conditions. VEGF and β-actin mRNA levels were detected by RT-PCR. FIG. 2B, Cells were treated as in FIG. 2A (0.1% or 2% O₂) followed by quantitative RT-PCR analysis of VEGF transcription; * p<0.05, **p<0.01, ***p<0.001, n=4.

FIG. 3 shows that erythropoeitin receptor (EPO-R) is expressed in SK-OV-3 cells, and EPO does not affect cell growth rates. FIG. 3A, RNA was extracted from SK-OV-3 cells and EPO-R mRNA was amplified using RT-PCR. Shown is an agarose gel of DNA marker (M, lane 1) and amplified EPO-R (Lane 2). FIG. 3B, Cells were treated for 16 h in normoxia (N) or 2% O₂ (hypoxia; H) followed by immunoblot analysis of cell lysates using an antibody recognizing EPO-R. C, Cells were seeded at low density and treated where indicated with 250 U/ml EPO. Treated media was replaced on day 2 and 5. Cell counts were measured by flow cytometry and values were normalized to day 0; n=3.

FIG. 4 shows that EPO inhibits HIF-1α induction by hypoxia in other cancer cell lines. MCF-7 cells were treated for 16 h with the indicated doses of EPO under normoxic and hypoxic (2% O₂) conditions. Cell lysates (25 μg) were analyzed by immunoblot using an antibody recognizing HIF-1α.

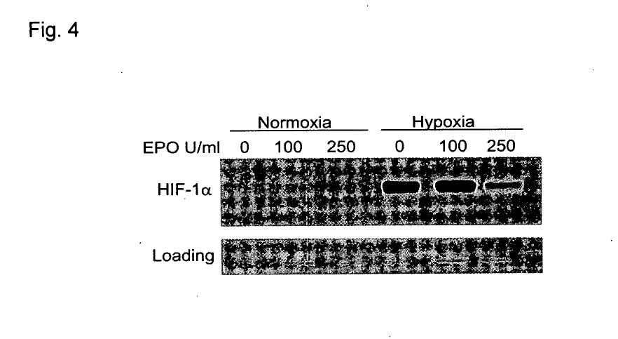

### DETAILED DESCRIPTION OF THE INVENTION

Recombinant human erythropoietin (Epoetin alfa, EPO) is used to combat anemia in patients treated for chronic kidney disease and in cancer patients undergoing chemotherapy (Fisher J W., Exp Biol Med (Maywood), 2003, 228:1-14). Given that erythropoietin expression is regulated by HIF-1α, we hypothesized that EPO may have an anti-angiogenic effect through negative feedback regulation of HIF-1α, resulting in reduced VEGF transcription. Here we present evidence that EPO inhibits hypoxia-induced HIF-1α stabilization and VEGF transcription in ovarian cancer cells without affecting cell growth rate. This effect is not restricted to ovarian cancer cells, suggesting that EPO may exert anti-angiogenic activity in a variety of cancer cells.

The invention encompasses the administration of EPO or EPO analogs, optionally along with other medicaments, which may provide a synergistic effect useful in the prevention and/or treatment of conditions that involve unwanted angiogenesis, such as cancer.

Accordingly, methods for inhibition of angiogenesis are provided, as are methods for treatment of cancer. Also provided are methods for reducing HIF-1α protein levels in a cell or tumor, and methods for reducing VEGF expression (e.g., by reduction of transcription), particularly the levels of HIF-1α and/or VEGF that are induced under conditions of hypoxia. Certain methods include the administration of an effective amount of at least one EPO or EPO analog formulated for administration to a subject. In other methods, cells are contacted with an effective amount of at least one EPO or EPO analog.

EPO as used herein is recombinant human erythropoietin (Epogen® or Procrit®). EPO molecules of other species also can be used.

EPO analogs include darbepoetin alfa (Aranesp®), polyethylene glycol (PEG)-EPO conjugates (e.g., U.S. Pat. No. 6,340,742), EPO molecules with additions, deletions or substitutions of amino acids (e.g., U.S. Pat. No. 5,888,772), EPO analog-human serum albumin fusions (e.g., U.S. Pat. No. 6,548,653), and the like. EPO analogs can be tested for suitability in the methods and compositions of the invention using standard angiogenesis assays and/or the methods described in the Examples herein.

When inhibition or reduction of certain parameters is described in response to treatment with at least one EPO or EPO analog the inhibition or reduction is at least about 10% of the parameter prior to the treatment. Preferably the inhibition or reduction is at least about 20%, more preferably at least about 25%, 30%, 35%, 40%, 45%, 50%, 55%, 60%, 65%, 70%, 75%, 80%, 85%, 90%, or even 95% or more.

As used herein, the term “angiogenesis” means the generation of new blood vessels into a tissue or organ. Under normal physiological conditions, humans or animals undergo angiogenesis only in very specific restricted situations. For example, angiogenesis is normally observed in wound healing, fetal and embryonal development and formation of the corpus luteum, endometrium and placenta. The term “endothelium” means a thin layer of flat epithelial cells that lines serous cavities, lymph vessels, and blood vessels. The term “endothelial inhibiting activity” means the capability of a molecule to inhibit angiogenesis in general and, for example, to inhibit the growth of bovine capillary endothelial cells in culture in the presence of fibroblast growth factor.

In accordance with the invention, EPO or EPO analogs are effective in treating diseases or processes that are mediated by, or involve, angiogenesis. The present invention includes method of treating an angiogenesis-mediated disease with an effective amount of EPO or EPO analogs. In particular, the diseases include, but are not limited to, cancers in which subjects have tumors. Tumors include solid tumors; blood born tumors such as leukemias; tumor metastases; benign tumors, for example hemangiomas, acoustic neuromas, neurofibromas, trachomas, and pyogenic granulomas; and pre-malignant tumors.

According to the invention, the terms “treating” and “treatment” include prophylaxis and therapy. When provided prophylactically, a treatment may be administered to a subject in advance of cancer (e.g., to a subject at risk of cancer) or upon the development of early signs of cancer in a subject. A prophylactic treatment serves to prevent, delay, or reduce the rate of onset of cancer or the appearance of symptoms associated with cancer. When provided therapeutically, a treatment may be administered at (or after) the onset of the appearance of symptoms of actual cancer. Therapy includes preventing, slowing, stopping, or reversing cancer or certain symptoms associated with cancer. In some embodiments, a treatment may serve to reduce the severity and duration of cancer or symptoms thereof. In some embodiments, treating a subject may involve halting or slowing the progression of cancer or of one or more symptoms associated with cancer. In some embodiments, treating a subject may involve preventing, delaying, or slowing the onset or progression of long-term symptoms associated with cancer. In some embodiments, treating a subject may involve complete or partial remission.

As used herein, a “subject” is a mammal, preferably a human, non-human primate, cow, horse, pig, sheep, goat, dog, cat or rodent. In all embodiments, human subjects are preferred. A “subject in need” and “subject in need of treatment” as used herein is a subject that is suspected of having cancer or has been diagnosed with cancer. A subject in need is also a subject at risk of having cancer as determined by associated risk factors including but not limited to smoking, family history, genetic predisposition, and external factors (for example environmental factors).

For human cancers, particular examples include, biliary tract cancer; bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease and Paget's disease; liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma, neurosarcoma, chondrosarcoma, Ewing sarcoma, malignant fibrous histocytoma, glioma, esophageal cancer, hepatoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor.

EPO or EPO analogs also can be used in the treatment of precancerous lesions such as epithelial precancerous lesions. An epithelial precancerous lesion is a lesion of epithelial cell origin that has a propensity to develop into a cancerous condition. An example is a precancerous skin lesion.

EPO or EPO analogs also can be used in combination with other compositions and procedures for the treatment of diseases. For example, a subject having one or more tumors may be treated conventionally with surgery, radiation or chemotherapy combined with EPO or EPO analogs and then EPO or EPO analogs may be subsequently administered to the patient to extend the dormancy of micrometastases and to stabilize any residual primary tumor. In some instances it may be preferable to administer the EPO or EPO analogs specifically to a site likely to harbor a metastatic lesion (that may or may not be clinically discernible at the time). A sustained release formulation implanted specifically at the site (or the tissue) where the metastatic lesion is likely to be would be suitable in these latter instances.

The EPO or EPO analogs of the invention can be administered concurrently with, or sequentially with, other antiangiogenesis molecules. Coadministration may be in the form of administration of a composition containing both kinds of antiangiogenic agents, or a plurality of compositions, each of which may contain one or more than one of the antiangiogenic agents.

An “antiangiogenic compound” or “antiangiogenesis molecule” as used herein is any molecule that inhibits capillary endothelial cell proliferation and/or migration and/or blood vessel ingrowth. Other antiangiogenesis molecules also can be administered to the subject, including, but not limited to endogenous angiogenesis inhibitors including PD 174073 and PD 166285 (Parke-Davis), SU5416 and SU6668 (Sugen), ZD 4190 and ZD 6474 (Zeneca), PTK 787 (also known as CGP79787 or ZK22584) (Novartis), Anti-VEGF mAb (Genentech), Anti-KDR mAb (ImClone), RPI 4610 (Ribozyme), TNP 470 (Abbott/TAP), AG 3340 (Agouron), Marimastat (British Biotech), Bay 12-9566 (Bayer), Neovastat (Aeterna), BMS 275291 (Bristol Myers-Squibb), CGS 27023A (Novartis), D1927 Chiroscience), D2163 (Chiroscience), Isoquinolines (Pfizer), Vitaxin (1×SYS), S-137 (Searle), S-836 (Searle), SM256 (Dupont), SG545 (Dupont), Angiostatin (EntreMed), Endostatin (EntreMed), Thalidomide (EntreMed), Squalamine (Magainin), CAI (National Cancer Institute), CM-101 (CarboMed), U-995 (Gwo-Chyang GMP), Combretastatin A-4 (Oxigene), platelet factor-4, vasostatin, thrombospondin, tissue inhibitors of metalloproteinases (TIMPs), STI412 (Sun and McMahon, Drug Discov. Today 5(8):344-353, 2000; Klohs and Hamby, Curr. Opin. Biotechnol. 10:544-549, 1999), fumagillin, non-glucocorticoid steroids and heparin and heparin fragments and antibodies to one or more angiogenic peptides such as α-FGF, β-FGF, VEGF, IL-8, and GM-CSF. Some of the foregoing may be administered in the form of nucleic acids encoding proteins; in each case the active agent is a protein and not the nucleic acid encoding the protein.

The invention may be used in the treatment of cancer. In these methods, an effective amount of EPO or EPO analog is administered to a subject having cancer, or in other instances a subject at risk of developing cancer. Other antiangiogenesis molecules also can be administered, as described herein. In addition, in certain embodiments of the invention, anticancer molecules are administered in combination with the antiangiogenesis molecules.

The compositions and methods of the invention in certain instances may be useful for replacing existing surgical procedures or drug therapies, although in most instances the present invention is useful in improving the efficacy of existing therapies for treating such conditions. Accordingly combination therapy may be used to treat the subjects. For example, the agent may be administered to a subject in combination with an anti-proliferative (e.g., an anti-cancer) therapy. Suitable anti-cancer therapies include surgical procedures to remove the tumor mass, chemotherapy or localization radiation. The anti-proliferative therapy may be administered before, concurrent with, or after treatment with the EPO or EPO analog in accordance with the invention. There may also be a delay of several hours, days and in some instances weeks between the administration of the different treatments, such that the EPO or EPO analog may be administered before or after the other treatment.

As an example, the EPO or EPO analog may be administered in combination with surgery to remove an abnormal proliferative cell mass. As used herein, “in combination with surgery” means that the agent may be administered prior to, during or after the surgical procedure. Surgical methods for treating epithelial tumor conditions include intra-abdominal surgeries such as right or left hemicolectomy, sigmoid, subtotal or total colectomy and gastrectomy, radical or partial mastectomy, prostatectomy and hysterectomy. In these embodiments, the EPO or EPO analog may be administered either by continuous infusion or in a single bolus or series of intermittent administrations. Administration during or immediately after surgery may include a lavage, soak or perfusion of the tumor excision site with a pharmaceutical preparation of the agent in a pharmaceutically acceptable carrier. In some embodiments, the agent is administered at the time of surgery as well as following surgery in order to inhibit the formation and development of metastatic lesions. The administration of the agent may continue for several hours, several days, several weeks, or in some instances, several months following a surgical procedure to remove a tumor mass.

The subjects can also be administered the agent in combination with non-surgical anti-proliferative (e.g., anti-cancer) drug therapy. In one embodiment, the agent may be administered in combination with an anti-cancer compound such as a cytostatic compound. A cytostatic compound is a compound (e.g., a nucleic acid, a protein, a small molecule) that suppresses cell growth and/or proliferation. In some embodiments, the cytostatic compound is directed towards the malignant cells of a tumor. In yet other embodiments, the cytostatic compound is one which inhibits the growth and/or proliferation of vascular smooth muscle cells or fibroblasts.

Suitable anti-proliferative drugs or cytostatic compounds to be used in combination with the agents of the invention include anti-cancer drugs. These anti-cancer agents may act by directly killing cells, such as cancer cells (i.e., direct action anti-cancer agents), or alternatively they may act by sensitizing cells to direct action anti-cancer agents (i.e., indirect action anti-cancer agents). Those of skill in the art will recognize the distinction and are familiar with agents of either class. Anticancer agents include, but are not limited to, the following sub-classes of compounds:

Antineoplastic agents such as: Acivicin; Aclarubicin; Acodazole Hydrochloride; Acronine; Adozelesin; Adriamycin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n 1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide.

Other anti-neoplastic compounds include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives (e.g., 10-hydroxy- camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflornithine; elemene; emitefur; epirubicin; epothilones including desoxyepothilones (A, R=H; B, R=Me); epithilones; epristeride; estramustine analogue; estrogen agonists; estrogen antagonists; etanidazole; etoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; R11 retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

Anti-cancer Supplementary Potentiating Agents: Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL. The compounds of the invention also can be administered with cytokines such as granulocyte colony stimulating factor.

Antiproliferative agent: Piritrexim Isethionate.

Radioactive agents: Fibrinogen I 125; Fludeoxyglucose F 18; Fluorodopa F 18; Insulin I 125; Insulin I 131; lobenguane I 123; lodipamide Sodium I 131; Iodoantipyrine I 131; Iodocholesterol I 131; Iodohippurate Sodium 1123; Iodohippurate Sodium I 125 Iodohippurate Sodium I 131; Iodopyracet I 125; Iodopyracet 1131; lofetamine Hydrochloride I 123; Iomethin I 125; lomethin I 131; lothalamate Sodium 1125; Iothalamate Sodium I 131; lotyrosine 1131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m Sulfur Colloid; Technetium Tc 99m Teboroxime; Technetium Tc 99m Tetrofosmin; Technetium Tc 99m Tiatide; Thyroxine 1125; Thyroxine I 131; Tolpovidone I 131; Triolein I 125; Triolein I 131.

The EPO or EPO analogs are delivered in effective amounts. In general, the term “effective amount” of EPO or EPO analogs refers to the amount necessary or sufficient to realize a desired biologic effect. Specifically, the effective amount is that amount that reduces the rate of or inhibits altogether angiogenesis, or that reduces HIF-1α protein levels in a cell or tumor, or that reduces VEGF levels (e.g., by reduction of transcription). For instance, when the subject bears a tumor having a blood supply, an effective amount is that amount which decreases or eliminates all together the blood supply to the tumor. Additionally, an effective amount may be that amount which prevents an increase or causes a decrease in new blood vessels, e.g., those vessels supplying a tumor.

The effective amount may vary depending upon whether the EPO or EPO analog is used alone or in combination with other therapeutics, or in single or multiple dosages. In some instances, it is envisioned that the combination of EPO or EPO analog with other therapeutic agents (which are themselves not EPO or EPO analog) can result in a synergism between the two compound classes, and thereby would require less of one or both compounds in order to observe the desired biologic effect. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject.

As mentioned above, the effective amount for any particular application can vary depending on such factors as the type of condition having unwanted angiogenesis being treated or prevented, the particular EPO or EPO analog being administered, the use of another antiangiogenesis agent, the size of the subject, or the severity of the disease or condition. One of ordinary skill in the art can empirically determine the effective amount of a particular EPO or EPO analog without necessitating undue experimentation.

Subject doses of the compounds described herein typically range from about 0.1 μg to 10 mg per administration, which depending on the application could be given hourly, daily, weekly, or monthly and any other amount of time therebetween. For example, in one regimen for treating cancer patients on chemotherapy with anemia, the recommended starting dose of EPO (Procrit®) for adults is 150 Units/kg subcutaneously three times a week. If the response is not satisfactory in terms of reducing transfusion requirements or increasing hematocrit after 8 weeks of therapy, the dose of Procrit can be increased up to 300 Units/kg three times a week. In some embodiments, however, doses of EPO or EPO analogs for the purposes of the invention may be used that are higher or lower than the typical doses described above.

For any EPO or EPO analog useful in the methods described herein, the therapeutically effective amount can be initially determined from animal models, e.g. animal models well known in the art. A therapeutically effective dose can also be determined from human data for EPO or EPO analogs which have been used in humans and for compounds which are known to exhibit similar pharmacological activities, such as other antiangiogenesis agents. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the EPO or EPO analog can be administered to a subject by any mode that delivers the EPO or EPO analog to a subject. “Administering” the pharmaceutical composition of the present invention may be accomplished by any means known to the skilled artisan. Some routes of administration include but are not limited to oral, intranasal, intratracheal, inhalation, ocular, vaginal, rectal, parenteral (e.g. intramuscular, intradermal, intravenous, intratumoral or subcutaneous injection) and direct injection.

For oral administration, the EPO or EPO analogs can be delivered alone without any pharmaceutical carriers or formulated readily by combining the active compound(s) with pharmaceutically acceptable carriers well known in the art. The term “pharmaceutically-acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray, from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds and optionally carrier compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The EPO or EPO analog may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The EPO or EPO analogs useful in the invention may be delivered in mixtures with additional antiangiogenesis agent(s). A mixture may consist of several antiangiogenesis agents in addition to the EPO or EPO analog.

A variety of administration routes are available. The particular mode selected will depend, of course, upon the particular molecules or other agents selected, the particular condition being treated and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration are discussed above.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

### EXAMPLES

Materials and Methods

Cell Culture Conditions. SK-OV-3 human ovarian cancer cells were maintained in McCoy's Media (Mediatech, Herndon, Va.) supplemented with 10% FBS (Gibco, Carlsbad, Calif.), Penicillin/Streptomycin (Gibco) and L-glutamine (Gibco), MCF-7 cells, a gift from Dr. Mercedes Rincon (The University of Vermont, Burlington, Vt.) were maintained in supplemented RPMI 1640 (Gibco). For 2% O₂ hypoxia exposure, cells were incubated at 2% O₂ by N₂ injection into a humidified CO₂ incubator (Form a, Marietta, Ohio). For 0.1% O₂ hypoxia exposure cells were incubated in the above incubator with injection of pre-mixed gas containing 5% CO₂, 95% N₂ and regulated by a ProOx oxygen controller (BioSpherix, Redfield, N.Y.). EPO (Epoetin Alfa, PROCRIT®) was obtained from Ortho Biotech, Bridgewater, N.J. Cell culture reagents were obtained from Gibco (Carlsbad, Calif.).

Semi-quantitative Reverse transcriptase-polymerase chain reaction (RT-PCR). RNA extraction was performed using the Trizol-chloroform method as described (Stevenson A S, et al., Exp Cell Res., 2001, 263:118-30). First strand cDNA was synthesized from 25 ng of total RNA by Sensiscript reverse transcriptase following the manufacturer's protocol (Qiagen, Valencia, Calif.). PCR reactions were carried out following the manufacturer's protocol (Invitrogen, Carlsbad, Calif.) with oligonucleotides recognizing human EPO-R (5′-GTGCTGGACAAATGGTTGCTG, SEQ ID NO:1; 3′-AGGAGGATGCTTCTGAGCCTTC, SEQ ID NO:2), 109 bp product), HIF-1α (Wellman T L, et al., Faseb J, 2004, 18:379-81), VEGF (Wellman T L, et al., Faseb J, 2004, 18:379-81), and β-actin (Wellman T L, et al., Faseb J, 2004, 18:379-81) (Qiagen). The resulting products were analyzed by ethidium bromide/agarose gel separation and quantified using a Fluor-S Multi-imager (Bio-Rad, Hercules, Calif.) and Quantity One™ analysis software.

Quantitative RT-PCR. Extraction of total RNA was performed as described above. First strand cDNA was synthesized from 1.5 μg of total RNA by Omniscript reverse transcriptase following the manufacturer's protocol (Qiagen, Valencia, Calif.). The PCR primers and Taqman probes for β2-microglobulin and VEGF were Assays-on-Demand products from Applied Biosystems (Foster City, Calif.). The PCR reactions were mixed and subsequent quantification of gene expression performed as described in (Dong Y, et al., Cancer Res 2004, 64:19-22). Temperature cycling and real-time fluorescence measurement were performed using an ABI prism 7700 Sequence Detection System (Applied Biosystems, Foster City, Calif.). The relative quantitation of gene expression was performed using the comparative C_{T}(ΔΔC_{T}) method (Dong Y, et al., Cancer Res 2004, 64:19-22).

Immunoblot. Cultured SK-OV-3 and MCF-7 cells were grown to ˜80% confluency. After treatments, cells were washed with ice-cold PBS, pH 7.4 and then harvested into hypotonic lysis buffer (HLB) containing: 25 mM Tris-HCL, pH 8, 2 mM MgCl₂, 5 mM KCl, 1 mM phenyl-methyl-sulfonamide, 20 μg/ml aprotinin, and 4 μg/ml leupeptin. Extracts were homogenized and centrifuged at 1,000 g to sediment the nuclear fraction. Nuclear pellets were washed, resuspended in 100 μl HLB and passed through a 26½ gauge syringe to shear DNA. Protein was determined by Bradford assay, and 25 μg from each sample separated by 8% SDS-PAGE. Proteins were transferred to nitrocellulose and analyzed by immunoblot using mouse monoclonal anti-HIF-1α (1:250; BD Biosciences, Bedford, Mass.) or rabbit polyclonal anti-EPO-R (1:200; Santa Cruz Biotechnology, Inc, Santa Cruz, Calif.) as described in Lounsbury et al. (Lounsbury K M, et al., J. Biol. Chem., 1994, 269:11285-90). Reactive bands were detected by chemiluminescence (LumiGLO, Kirkegaard & Perry, Gaithersburg, Md.) and quantified using Quantity One™ analysis software.

Immunofluorescence. Cultured SK-OV-3 cells were grown to ˜80% confluency on glass coverslips in 6-well culture dishes. After treatment, the cells were washed with ice-cold PBS, fixed with 4% paraformaldehyde in PBS for 15 min, and analyzed by immunofluorescence as described (Wellman T L, et al., Faseb J, 2004, 18:379-81) using monoclonal mouse anti-HIF-1α (1:200; BD Biosciences, Bedford, Mass.) and CY3-anti-mouse IgG (1:500; Jackson IR Labs). Cells were counterstained with YOYO-1 (1:10,000; Molecular Probes) containing 250 μg/ml RNase A for 30 min at 37° C. After being washed extensively with PBS, coverslips were mounted with Aqua Poly/Mount (Polysciences, Inc., Warrington, Pa.). Immunofluorescence was detected using a Bio-Rad 1000 laser scanning confocal microscope with a 40× objective.

Flow cytometry. Cells were seeded at low density, allowed to grow for 24 h then serum starved (0.1% FBS) 24 h. After serum starving, cells were placed in either normoxia or hypoxia and treated with or without 250 U/ml EPO for six days. Treatment media was replenished on days 2 and 5. On Days 0 (24 h after serum starving), Day 1, Day 3 and Day 6 cells were harvested for flow cytometry analysis. Cells were trypsinized, resuspended in PBS and centrifuged at 1200 g for 3 min. Cells were resuspended in 2% BSA in PBS. Each sample was counted twice for 60 s on an EPICS® XL/XL-MCL Flow Cytometry System with 488 nm Argon laser.

Statistical Analysis. One-way ANOVA was performed on all data. A Kruskal-Wallis adjustment was used where necessary. All pairwise multiple comparisons were assessed using the Newman-Keuls method of ANOVA.

Results and Discussion

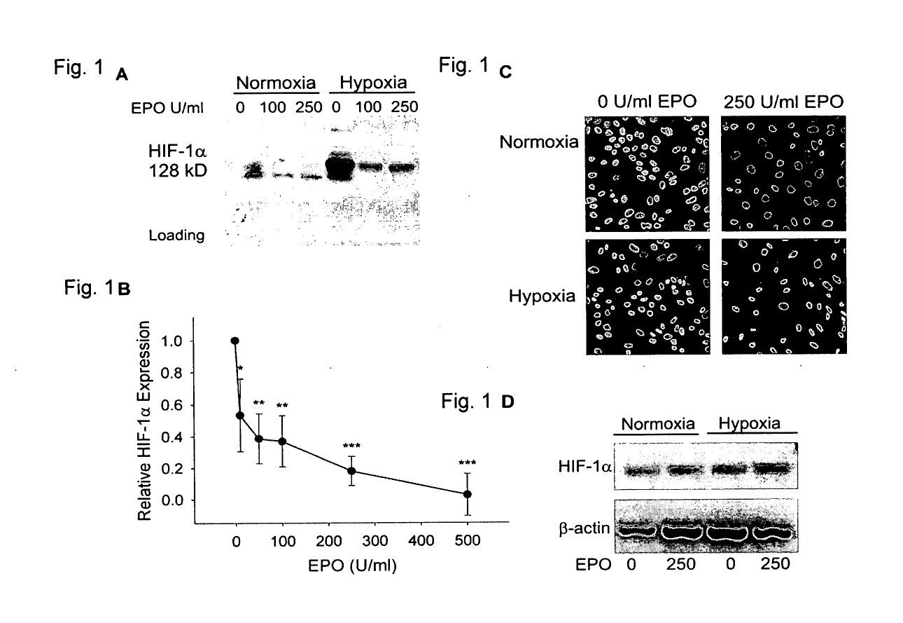

EPO prevents HIF-1α stabilization by hypoxia in ovarian cancer cells. To test our hypothesis that EPO exerts negative feedback on HIF-1α, SK-OV-3 cells were treated with EPO under either hypoxic or normoxic conditions, and then HIF-1α was detected in nuclear extracts by immunoblot or in fixed cells by immunofluorescence. EPO significantly decreased HIF-1α levels induced by hypoxia (2% or 0.1% O₂) at doses as low as 10 U/ml without altering normoxic levels of HIF-1α (FIG. 1A,B). Immunofluorescence confirmed that EPO did not reduce HIF-1α levels due to nuclear export (FIG. 1C). Furthermore, there was no effect of EPO on HIF-1α mRNA (FIG. 1D), suggesting that the reduction of HIF-1α is likely caused by protein destabilization or decreased translation rather than by reduction of HIF-1α transcription. The inhibitory effect of EPO on HIF-1α protein was selective to the extent that exposure to VEGF did not affect HIF-1α stabilization by hypoxia (Hale and Lounsbury, unpublished observation).

Based on the known growth factor activity of erythropoietin (Fisher J W., Exp Biol Med (Maywood), 2003, 228:1-14), these results are not particularly expected. Several reports have shown that growth factors and cytokines upregulate HIF-1α protein and transcriptional activity (Zhong H, et al., Cancer Research, 2000, 60:1541-1545; Jung Y J, et al., FASEB J., 2003, 14:2115-2117; Feldser D, et al., Cancer Res., 1999, 59:3915-8). However, unlike other growth factors and cytokines, erythropoietin is fundamentally regulated by HIF-1α, thus increasing the likelihood that it may exert a negative feedback role in its regulation.

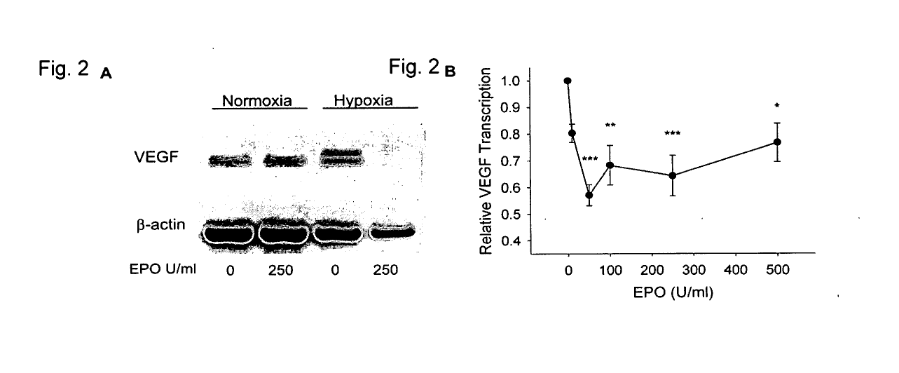

EPO treatment decreases VEGF transcription in SK-OV-3 ovarian cancer cells. HIF-1α is implicated in cancer progression through its transcriptional activation of pro-angiogenic factors such as VEGF. Because of the existence of multiple forms of HIF-1α, some of which are likely modified by ubiquitin or phosphorylation and are not transcriptionally functional, it was possible that a reduction in HIF-1α protein levels by EPO may not translate into a decrease in VEGF transcription (Kallio P J, et al., J Biol. Chem., 1999, 274:6519-25; Alfranca A, et al., Mol Cell Biol., 2002, 22:12-22). For this reason, it was important to measure EPO's ability to inhibit HIF-1α function. The impact of EPO on hypoxia-induced VEGF transcription was determined using semi-quantitative and quantitative RT-PCR analysis. EPO significantly reduced hypoxia-induced VEGF transcription in a dose-dependent fashion (FIG. 2). Although the dose response effects paralleled those on HIF-1α protein, the inhibition was not as extensive, suggesting that the VEGF transcription response may have a HIF-1α-independent component. We and others have observed similar results, leading to the proposal that VEGF transcription can occur through mechanisms independent of HIF-1α transcriptional regulation (Wellman T L, et al., Faseb J, 2004, 18:379-81; Mizukarni Y, et al., Cancer Res., 2004, 64:1765-72).

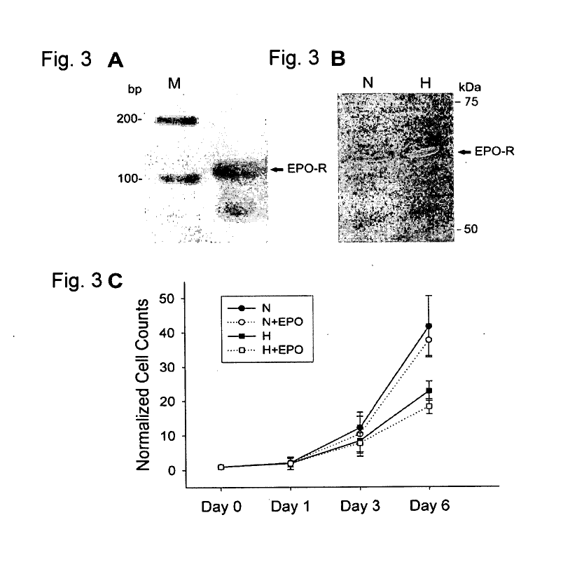

EPO-R is expressed in SK-OV-3 ovarian cancer cells. To verify that the observed effects of EPO on HIF-1α and VEGF could be mediated through the erythropoietin receptor (EPO-R), the expression of EPO-R was measured at the mRNA level using RT-PCR and at the protein level by immunoblot. As shown in FIG. 3, EPO-R mRNA and protein were both readily detected, and hypoxia had no apparent effect on the level of EPO-R protein. EPO-R has been identified in other cancer cell lines and in tumor tissues (Acs G, et al., Cancer Res., 2001, 61:3561-3565; Yasuda Y, et al., Carcinogenesis, 2003, 24:1021-1029). Our results corroborate these studies and further indicate that the opportunity exists for a signaling pathway between EPO-R and HIF-1α in ovarian cancer cells.

EPO does not affect the growth rate of SK-OV-3 ovarian cancer cells. The EPO-R signals through the Janus kinase (JAK/STAT) pathway, which is associated with increased growth and survival of cancer cells (Santos S C, et al., Oncogene, 2001, 20:2080-90). To determine if the EPO effects on angiogenic regulators were regrettably countered by increased cell growth, we used flow cytometry and found that treatment of SK-OV-3 cells with EPO did not increase the rate of cell growth under either normoxic or hypoxic conditions at any time point tested (FIG. 3C). As expected, hypoxia significantly abrogated the rate of cell growth (Schmaltz C, et al., Molecular and Cellular Biology, 1998, 18:2845-2854), but interestingly, treatment with EPO slightly enhanced the inhibitory effect.

There has been considerable debate over whether it is detrimental to administer EPO to cancer patients receiving chemotherapy. Cancer patients undergoing chemotherapy who have received EPO exhibit a trend toward improved survival (Littlewood T J, et al., J. Clinical Oncol., 2001, 19:2865-2874), however a recent report showed that exogenous EPO may facilitate the growth of tumors and contribute to a decrease in survivability. In addition, molecular studies have implicated high levels of erythropoietin and erythropoietin receptor (EPO-R) in progression of tumor growth (Acs G, et al., Cancer Res., 2001, 61:3561-3565; Yasuda Y, et al., Carcinogenesis, 2003, 24:1021-1029). Our results do not indicate an effect of EPO on the growth rate of SK-OV-3 human ovarian cancer cells. However the potential for unfavorable effects of EPO encourage further studies defining the scope of the observed effect and the mechanisms underlying HIF-1α inhibition by EPO.

Reduction of HIF-1α by EPO is not limited to ovarian cancer cells. To explore the negative feedback of EPO on HIF-1α in other cancer cell types, we tested the effect of EPO on hypoxia-induced HIF-1α in the breast cancer cell line, MCF-7. Unlike SK-OV-3 cells, MCF-7 cells had undetectable levels of HIF-1α in normoxia. Similarly to SK-OV-3 cells, EPO efficiently inhibited the HIF-1α stabilization induced by hypoxia, suggesting that the negative feedback of EPO on HIF-1α is not limited to ovarian cancer cells. Angiogenesis is critical for the growth and metastasis of multiple tumor types (Semenza G L., Nature Reviews Cancer, 2003, 3:721-732). Thus, these results broaden the potential impact that EPO administration may have in reducing angiogenesis in a variety of cancers and encourage pursuit of both the molecular mechanism of EPO action as well as the effect of EPO on tumor angiogenesis in vivo.

Together these studies reveal a novel approach to inhibiting angiogenesis through our discovery that hypoxia-induced VEGF transcription is disrupted by administration of EPO in ovarian cancer cells. These anomalous observed effects of EPO on VEGF transcription are mediated by inhibition of HIF-1α protein levels, provoking future investigation of the underlying molecular mechanism. Furthermore, since EPO does not exert measurable effects on cell growth rates during normoxia or hypoxia, we suggest that EPO has the potential to inhibit angiogenesis without increasing tumor cell growth in ovarian cancer.

### EQUIVALENTS

It should be understood that the preceding is merely a detailed description of certain preferred embodiments. It therefore should be apparent to those of ordinary skill in the art that various modifications and equivalents can be made without departing from the spirit and scope of the invention. It is intended that the invention encompass all such modifications within the scope of the appended claims.

All references, patents and patent applications and publications that are cited or referred to in this application are incorporated in their entirety herein by reference.

1. A method of treating cancer in a subject in need of such treatment comprising administering to the subject an amount of erythropoietin (EPO) or EPO analog effective to inhibit angiogenesis in a tumor in the subject.

2. The method of claim 1, wherein the tumor is a breast cancer, an ovarian cancer, a colon cancer, a brain cancer, a cervical cancer, a lung cancer, a hematological neoplasm, a liver cancer, a lymphoma, a pancreatic cancer, a prostate cancer, a sarcoma, or a skin cancer.

3. The method of claim 1, wherein the tumor is a solid tumor growth, a tumor metastasis, or a precancerous lesion.

4. The method of claim 1, wherein the EPO or EPO analog is administered locally.

5. The method of claim 1, wherein the EPO or EPO analog is administered systemically.

6. The method of claim 1, wherein the subject is otherwise free of symptoms calling for treatment with EPO or an EPO analog.

7. The method of claim 1, wherein the EPO or EPO analog is administered in combination with one or more anti-cancer compounds.

8. The method of claim 1, wherein the EPO or EPO analog is administered in combination with surgery to remove the tumor.

9. The method of claim 1, wherein the EPO or EPO analog is administered to a patient who has had surgery to remove the tumor.

10. The method of claim 1, wherein the EPO or EPO analog is administered in combination with one or more anti-angiogenic compounds.

11. A method for reducing hypoxia-induced HIF-1αprotein levels or VEGF expression, comprising
contacting a cell with an amount of erythropoietin (EPO) or EPO analog effective to reduce hypoxia-induced HIF-1α protein levels or VEGF expression.

12. The method of claim 11, wherein the EPO or EPO analog inhibits hypoxia-induced HIF-1α stabilization, increases HIF-1α destabilization, and/or decreases HIF-1α translation or inhibits VEGF transcription or translation.

13. The method of claim 11, wherein the HIF-1α protein levels or VEGF expression levels are reduced at least about 50%.

14. A method for reducing hypoxia-induced HIF-1α protein levels or VEGF expression in a subject, comprising
administering to the subject an amount of erythropoietin (EPO) or EPO analog effective to reduce hypoxia-induced HIF-1α protein levels or VEGF expression.

15. The method of claim 14, wherein the subject is otherwise free of symptoms calling for treatment with EPO or an EPO analog.

16. The method of claim 14, wherein the EPO or EPO analog is administered locally.

17. The method of claim 14, wherein the EPO or EPO analog is administered systemically.

18. The method of claim 14, wherein the EPO or EPO analog inhibits hypoxia-induced HIF-1α stabilization, increases HIF-1α destabilization, and/or decreases HIF-1α translation or inhibits VEGF transcription or translation.

19. The method of claim 14, wherein the HIF-1α protein levels or VEGF expression levels are reduced at least about 50%.

20-28. (canceled)

29. A pharmaceutical preparation comprising:
erythropoietin (EPO) and/or at least one EPO analog,
at least one anti-cancer compound and/or at least one anti-angiogenesis compound, and
a pharmaceutically acceptable carrier.

30. A kit comprising
a first container housing erythropoietin (EPO) and/or at least one EPO analog, and
a second container housing at least one anti-cancer compound and/or at least one anti-angiogenesis compound.

31-32. (canceled)
